(19) United States
(12) Patent Application Publication
(10) Pub. No.: US 2025/0282836 A1
(43) Pub. Date: Sep. 11, 2025

(54) Lipocalin 10 and its Truncated Protein as Therapeutic Agents for Inflammation-Induced Organ Dysfunction

(71) Applicant: University of Cincinnati, Cincinnati, HI (US)

(72) Inventors: Guo-Chang Fan, Cincinnati, OH (US); Xiaohong Wang, Blue Ash, OH (US); Yutian Li, Mason, OH (US)

(73) Assignee: University of Cincinnati, Cincinnati, OH (US)

(21) Appl. No.: 18/276,122

(22) PCT Filed: Feb. 7, 2022
(86) PCT No.: PCT/US22/15480

§ 371 Date: Aug. 7, 2023

Related U.S. Application Data
(60) Provisional application No. 63/146,321, filed on Feb. 5, 2021.
(60) Provisional application No. 63/278,740, filed on Nov. 12, 2021.

(51) Int. Cl. C07K 14/47; A61K 38/00

(52) U.S. Cl. CPC C07K 14/47; A61K 38/00

(57) ABSTRACT

A method of reducing the risk of a sepsis-induced vascular leak, tissue edema or organ dysfunction is provided. The method involves administering an effective amount of a composition selected from the group consisting of Lipocalin 10 (SEQ ID NO: 1), a truncated Lipocalin 10 (Lcn10) protein having the amino acid sequence SEQ ID NO: 2, Lcn 10-expressing vectors for full length/truncated Lcn10, or combinations thereof to the subject, The method is also useful for reducing the risk of a heart attack-induced cardiac dysfunction, atherosclerosis, inflammatory bowel disease or diabetes-induced cardiomyopathy.

### CROSS REFERENCE TO RELATED APPLICATIONS

This application claims priority to PCT Application PCT/US22/15480, filed Feb. 7, 2022, U.S. Provisional Application Ser. No. 63/146,321, filed Feb. 5, 2021, and U.S. Provisional Application Ser. No. 63/278,740, filed Nov. 12, 2021, which applications are hereby incorporated by reference in their entirety.

### STATEMENT REGARDING FEDERALLY FUNDED RESEARCH OR DEVELOPMENT

This invention was made with government support under grants R01-GM132149 and R01-HL160811, awarded by the National Institutes of Health. The U.S. Government has certain rights in the invention.

### TECHNICAL FIELD

The present invention relates generally to treatments to prevent the risk of sepsis.

### BACKGROUND OF THE INVENTION

Despite recent advances in antibiotic therapy and supportive critical care, sepsis remains a leading cause of death in intensive care units. Currently, the increased vascular permeability is well-recognized to be responsible for sepsis-triggered organ failure and patient mortality. This is particularly the case in the lungs where capillary leak induces lung edema, leading to acute respiratory distress syndrome (ARDS). However, whether vascular permeability increases in the heart that contributes to cardiac dysfunction during sepsis is not well-studied. Recently, we and others showed a positive connection between vascular leakage, cardiac depression, and mortality during sepsis. Nonetheless, the mechanisms underlying sepsis-induced cardiac capillary leak remains obscure. In general, vascular barrier integrity is maintained by junctional complexes including tight junctions and adherens junctions which are anchored to the actin cytoskeleton between endothelial cells (ECs). Upon sepsis conditions, proinflammatory factors can disrupt EC barrier integrity by either altering protein levels of junctional molecules or disturbing actin dynamics, leading to paracellular gap formation. At present, a variety of mediators (i.e., Rac1/RhoA) have been identified to play a critical role in regulating vascular permeability. However, to date, no specific treatment targeting the vascular leak in sepsis is yet available. Thus, a deeper understanding of mediators and their associated mechanisms in sepsis-elicited vascular leak is of great importance for the development of future therapeutic strategies.

### SUMMARY OF THE INVENTION

One embodiment of the present invention addresses this need by providing a method of reducing the risk of a sepsis-induced vascular leak, tissue edema or organ dysfunction in a subject. The method involves administering an effective amount of a composition selected from the group consisting of Lipocalin 10 (SEQ ID NO: 1), a truncated Lipocalin 10 (Lcn10) protein having the amino acid sequence SEQ ID NO: 2, Lcn10-expressing vectors for full length/truncated Lcn10, or combinations thereof to the subject. In one embodiment, the method is used to reduce the risk of a sepsis-induced vascular leak. In another embodiment, the method is used to reduce the risk of tissue edema. In one embodiment, the method is used to reduce the risk of organ dysfunction. In one embodiment, the subject is administered Lipocalin 10 (SEQ ID NO:1). In another embodiment, the subject is administered a truncated Lipocalin 10 (Lcn10) protein having the amino acid sequence SEQ ID NO: 2. In one embodiment, the subject is administered with Lipocalin 10 (SEQ ID NO:1) or a truncated Lipocalin 10 (SEQ ID NO: 2) at a dosage of 50-200 ng/g body weight. In another embodiment, the subject is administered with Lipocalin 10 (SEQ ID NO: 1) or a truncated Lipocalin 10 (SEQ ID NO: 2) via vein injection.

In another embodiment of the present invention, a method of reducing the risk of a heart attack-induced cardiac dysfunction, atherosclerosis, inflammatory bowel disease or diabetes-induced cardiomyopathy in a subject is provided. The method involves administering an effective amount of a composition selected from the group consisting of Lipocalin 10 (SEQ ID NO: 1), a truncated Lipocalin 10 (Lcn10) protein having the amino acid sequence SEQ ID NO: 2, Lcn10-expressing vectors for full length/truncated Lcn10, or combinations thereof to the subject. In one embodiment, the method is used to reduce the risk of a heart attack-induced cardiac dysfunction. In another embodiment, the method is used to reduce the risk of atherosclerosis. In one embodiment, the method is used to reduce the risk of inflammatory bowel disease. In another embodiment, the method is used to reduce the risk of diabetes-induced cardiomyopathy. In one embodiment, the subject is administered Lipocalin 10 (SEQ ID NO:1). In another embodiment, the subject is administered a truncated Lipocalin 10 (Lcn10) protein having the amino acid sequence SEQ ID NO: 2.

In another embodiment of the present invention, a pharmaceutical composition comprising a truncated Lipocalin 10 protein having the amino acid sequence SEQ ID NO: 2 is provided. In one embodiment, the composition is for use in a method of reducing the risk of a sepsis-induced vascular leak, tissue edema or organ dysfunction in a subject. In another embodiment, the composition is for use in a method of reducing the risk of a heart attack-induced cardiac dysfunction, atherosclerosis, inflammatory bowel disease or diabetes-induced cardiomyopathy in a subject.

### BRIEF DESCRIPTION OF THE DRAWINGS

The foregoing summary, as well as the following detailed description of preferred embodiments of the application, will be better understood when read in conjunction with the appended drawings.

FIG. 1A is a schematic showing cofilin-mediated actin dynamics in endothelial cells upon resting and sepsis conditions. TJ=tight junction; AJ=adherence junction.

FIG. 1B is a graph showing expression levels of LIMK2 and Ssh1 in blood samples collected from 6 cohorts of sepsis patients and healthy donors.

FIG. 2 is a blot showing genotyping of a global Lcn10-KO mouse model. +/+: wild-type (WT); +/−: heterozygous; −/−: homozygous (KO).

FIGS. 3A and 3B are an image and a graph showing the generation of endothelial cell-specific Lcn10-transgenic mice (EC-Lcn10-Tg). FIG. 3A is a diagram of a transgenic vector; and FIG. 3B is RT-qPCR results showing that the mRNA levels of Lcn10 were only increased in cardiac ECs (C-ECs) but not in cardiac fibroblasts or myocytes. (n=4, *, p<0.01 vs. WTs).

FIGS. 4A-4D are a series of graphs showing dynamic alterations of Lcn10 expression in mouse hearts following LPS injection (FIG. 4A) occurs only in cardiac endothelial cells but not in either fibroblasts or cardiomyocytes (FIG. 4B). Similar results were observed in mouse hearts (FIG. 4C) following CLP surgery and in cardiac ECs (FIG. 4D) (n=4-9, *, p<0.05 vs. Oh).

FIGS. 5A-5D are a series of images and graphs showing: (FIG. 5A) Representative echocardiography images and their analysis reveal the significant reduction of EF % (FIG. 5B) and FS % (FIG. 5C) in LPS-treated Lcn10-KO mouse hearts, compared to LPS-WTs (n=5-7, *, p<0.05 vs. LPS-WTs). (FIG. 5D) The survival rate is greatly lower in Lcn10-KO mice than WT-mice following LPS injection. (n=10, *, p<0.05 vs. WTs).

FIG. 6 is a volcano plot graph summarizing the RNA-seq. results of gene expression profiles in Ad.Lcn10- vs. Ad.GFP-infected ECs.

FIGS. 7A-7C are a series of graphs showing (FIG. 7A) Exogenous addition of rLcn10 and (FIG. 7B) endogenous elevation of Lcn10 by adenoviral vectors could upregulate both LRP2 and Ssh1 expression. (FIG. 7B, FIG. 7C) Knockdown of LRP2 could block Lcn10-induced elevation of Ssh1 in either (FIG. 7B) Ad.Lcn10-ECs or (FIG. 7C) rLcn10-treated ECs, compared to respective controls. *, p<0.05, n=4-6; ns: not significant.

FIGS. 8A and 8B are a pair of graphs showing that adenovirus vector-mediated overexpression of Lcn10 protects against LPS-caused EC leakage, which is dependent on the Ssh1 signaling, as measured by (FIG. 8A) FITC-dextran and (FIG. 8B) EB-albumin flux. (n=5, *p<0.05; ns, not significant).

FIG. 9 is a scheme showing the Len10-induced reduction of vascular leakage via the LRP2-Ssh1-Cofilin signaling during sepsis.

FIGS. 10A-10C are a series of images and graphs showing that treatment of ECs with rLcn10 greatly protects against endothelial leakage, as evidenced by increases TEER (FIG. 10A) and decreases leakage of FITC-dextran (FIG. 10B) and EB-albumin (FIG. 10C), compared to BSA-controls (n=4; *, p<0.05).

FIGS. 11A-11C are a series of graphs showing that the knockdown of Ssh1 by siRNAs greatly offset rLcn10-induced protection against vessel leakage, as evidenced by the increases of TEER (FIG. 11A), decreases of FITC (FIG. 11B) and EB (FIG. 11C) flux, (n=4-6; *, p<0.05; ns, not significant).

FIGS. 12A-12E are a series of graphs showing the expression levels of Len10 in tissues of wild-type mice as well as in the blood and spleen of septic mice.

FIGS. 13A-13H are a series of graphs showing that Len10 deficiency increases inflammatory response locally and systemically in sepsis. Serum and peritoneal lavage fluid were harvested at 16 h after CLP-operation. The pro-inflammatory cytokines TNF-α (FIG. 13A, FIG. 13E), IL-6 (FIG. 13B, FIG. 13F) and MCP-1 (FIG. 13C, FIG. 13G) and anti-inflammatory cytokine IL-10 (FIG. 13D, FIG. 13H) were measured using ELISA kits. #, P<0.05; n=6-8 mice per group.

FIGS. 14A-14E are a series of images and graphs showing that Lcn10 deficiency attenuates bacterial clearance and suppressed phagocytic capability of peritoneal macrophages (PMs) from septic mice.

FIGS. 15A-15F are a series of graphs showing the dynamic expression of Lcn10 in septic hearts and septic endothelial cells. CLP: cecal ligation and puncture.

FIGS. 16A-16H are a series of graphs showing that knockdown of Lcn10 augments the permeability in mouse cardiac endothelial cells (MCECs).

FIGS. 17A-17G are a series of images and graphs showing that overexpression of full-length Lcn10 reduces LPS- or TNFalpha-caused endothelial cell leakage.

FIGS. 18A and 18B are a series of graphs showing expression levels of Lcn10 in different tissues of wild type (WT) mouse and efferocytic macrophages.

FIGS. 19A-19E are a series of graphs showing that the absence of Len10 exacerbates I/R-induced cardiac dysfunction and cardiac damage.

FIGS. 20A-20D are a series of graphs showing that addition of recombinant Lcn10 protein increases macrophage efferocytosis in vitro through the upregulation of MerTK.

FIGS. 21A-21E are a series of graphs showing that the overexpression of Lcn10 in macrophgges upregulates anti-inflammatory genes.

FIG. 22 is a series of graphs showing the reduced Len10 expression in macrophages upon different metabolic stress conditions.

FIGS. 23A and 23B are a pair of graphs showing that the overexpression of Lcn10 in Raw264.7 cells reduced inflammatory response to LPS, evidenced by reduced levels of IL-6 (FIG. 23A) and TNF-alpha (FIG. 23B).

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention has found the therapeutic potential of Lcn10 protein as a novel regulator of vascular barrier integrity and as a new protector against sepsis-induced organ failure. This invention provides new strategies to inhibit sepsis-induced vascular hyperpermeability and as a consequence, minimize fluid resuscitation and tissue edema. This novel approach can improve the survival of septic patients.

Lipocalin 10 (Lcn10) is a secreted protein that is a member of lipocalin family. Of specific interest, Lcn10 is highly expressed in the heart, lymph node, spleen and thyroid. Data has shown that Lcn10 was significantly down-regulated in the hearts of both endotoxin LPS- and cecal ligation-puncture (CLP)-treated mice, compared to their controls. Interestingly, further analysis of Lcn10 expression in different cell types isolated from LPS- and CLP-treated hearts showed that reduction of Lcn10 occurred only in cardiac ECs rather than in fibroblasts or cardiomyocytes. These compelling data indicate that Len10 is involved in sepsis-induced cardiac vascular leak.

Using a global Lcn10-knockout (KO) mouse model, the present invention found that deficiency of Lcn10 significantly increased vascular permeability, which correlated with more severe cardiac depression and higher mortality following LPS challenge, compared to LPS-treated wild-type (WT) mice. By contrast, in vitro overexpression of Lcn10 in ECs showed a greater resistance to LPS-caused monolayer leak compared to control cells. An initial mechanistic analysis by RNA-sequencing and RT-qPCR showed that both endogenous and exogenous elevation of Lcn10 in ECs could significantly upregulate slingshot homolog 1 (Ssh1) expression. Ssh1 is a phosphatase known to dephosphorylate and activate Cofilin, a key actin-binding protein that plays an essential role in controlling actin filament re-arrangement. Importantly, knockdown of Ssh1 in ECs by siRNA greatly offset Lcn10-induced reduction of monolayer permeability upon LPS insult. Thus, based on these data, the inventors have found that Lcn10 is critical for protecting against sepsis-induced cardiovascular leak via the activation of the Ssh1-Cofilin pathway.

To define the precise role of Lcn10 in vascular permeability during polymicrobial sepsis, a global Lcn10-KO mouse model can be utilized to test whether Lcn10-deficient mice are sensitive to sepsis-induced vascular leak, cardiac dysfunction, and death. By contrast, EC-specific Lcn10-transgenic (Tg) mice can be used to test if EC-specific elevation of Lcn10 protects against sepsis-triggered vascular leak and heart injury, leading to improved myocardial function and survival rate. Polymicrobial sepsis can be induced by cecal ligation and puncture (CLP) surgery, and animal survival can be monitored over time. Cardiovascular leakage, edema, leukocyte infiltration and cardiac function can then be measured. This helps to evaluate Len10 as a novel anti-sepsis mediator, which enhances vascular barrier integrity during sepsis.

Lcn10 may have autocrine effects on EC function during sepsis. Importantly, our data shows that the addition of recombinant Lcn10 protein (rLcn10) to cultured ECs resulted in a reduced monolayer leakage upon LPS insult. Injection of the rLcn10 protein into septic mice can suppress vascular leak and cardiac edema, improve myocardial function as well as animal survival outcomes.

Further, the present invention can help identify the mechanism by which Lcn10-elicited inhibition of cardiovascular leak is dependent on Ssh1-mediated actin dynamics. We have found that Lcn10 can upregulate Ssh1 expression in ECs, and knockdown of Ssh1 greatly reduced Lcn10-elicited protection against EC monolayer leak upon LPS exposure. Given that Ssh1 is a key regulator of actin dynamics in ECs, this indicates that Lcn10-mediated inhibition of sepsis-induced vascular leak is dependent on Ssh1-mediated actin filament reorganization.

### Lcn10 as Novel Regulator of Sepsis-Caused Microvascular Endothelial Leakage

Sepsis is initiated by an uncontrolled immune response to a local severe infection. Prompt antibiotic therapy and adequate intravenous fluid therapy are essential for the treatment of septic patients to reduce mortality. However, fluid overload can be harmful, because vascular leakage is increased during sepsis, which in turn causes hypoperfusion, tissue edema and finally, loss of organ function (e.g. heart failure). Therefore, therapeutic interventions aimed at reducing vascular leak could be an effective treatment option against sepsis. Unfortunately, no therapies targeting the increased vascular permeability in sepsis have been successful thus far, indicating the need to understand better the mediators and mechanisms involved in sepsis-triggered endothelial dysfunction. At present, most prior work has focused on the sepsis-induced pulmonary capillary hyperpermeability that results in severe lung edema and acute respiratory distress syndrome. Few studies have investigated coronary vascular leakage, which is a major cause of heart failure and death in human patients with septic shock.

Of the known lipocalin (Lcn) family members, only Lcn2 (also called neutrophil-gelatinase associated lipocalin, NGAL) has been well characterized for its role in cancer, infectious disease and metabolic disorders. Interestingly, recent RNA-sequencing analysis of peripheral blood collected from sepsis patients showed that Lcn2 expression was significantly elevated in sepsis non-survivals versus survivals. By contrast, Lcn10 expression was greatly reduced in sepsis non-survivals, compared to survivals.

Our data shows that expression levels of Lcn10 were greatly reduced in mouse hearts following either LPS treatment or cecal ligation and puncture (CLP) surgery. Of interest, such reduction of cardiac Lcn10 occurred only in endothelial cells (ECs), but not in either fibroblasts or cardiomyocytes. Furthermore, Lcn10-knockout (Lcn10-KO) mice exhibited higher mortality rate than control wild-type (WT) mice following endotoxin LPS challenge (n=10, p<0.05). Remarkably, LPS-treated Lcn10-KO mice revealed greater cardiac dysfunction, which was accompanied by the increased vascular leakage, compared to LPS-WTs. Importantly, in vitro overexpression of Len10 in mouse cardiac ECs significantly reduced monolayer permeability, compared to control cells, following LPS insult. Together, these data prompted us to test whether ablation of Lcn10 is sensitive, whereas elevation of Lcn10 in ECs is resistant, to vascular leak and cardiac dysfunction during polymicrobial sepsis induced by CLP surgery.

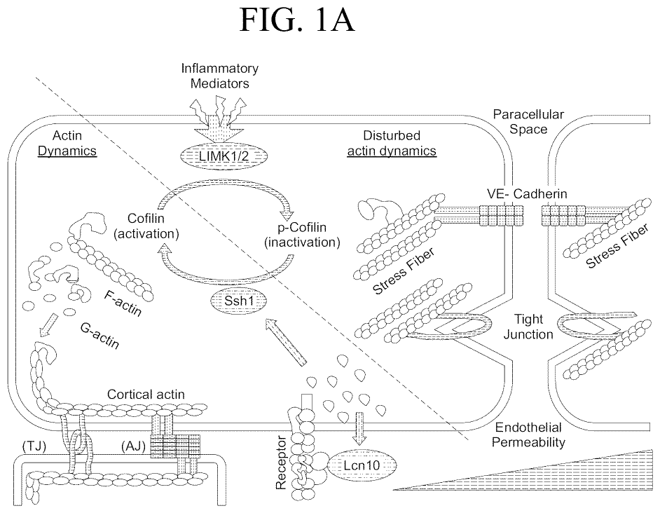

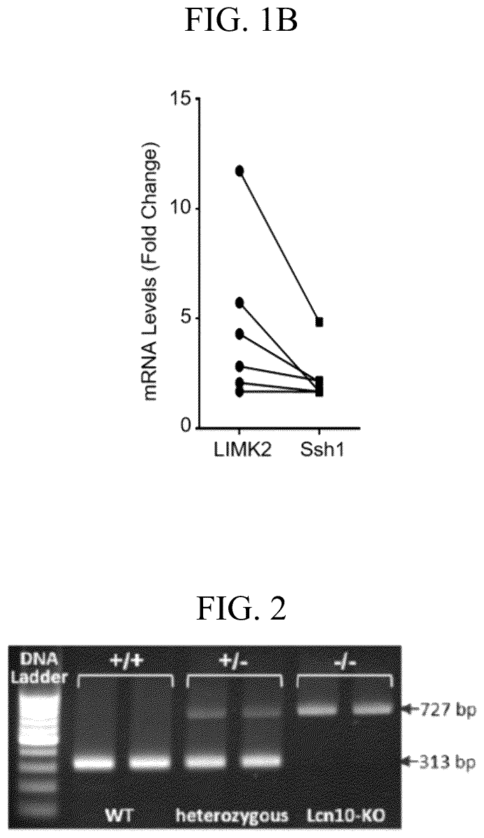

Cofilin-mediated actin filament dynamics in endothelial permeability and the mechanism underlying Lcn10-controlled vascular permeability. The current paradigm states that the increase of paracellular endothelial leakage is driven, on the one hand, by the generation of the centripetal contractile forces and, on the other hand, by the loss of junctional integrity, provided mostly by the tight and adherence junctions. Both endothelial contraction and the maintenance of junctional integrity depend on actin filament reorganization (FIG. 1A). Notably, there are two forms of actin known to exist in ECs including monomeric globular actin (G-actin) and polymerized filamentous actin (F-actin). Under resting conditions, a subset of G-actin monomers assemble along the cell periphery as cortical actin filaments, which connect to transmembrane junction proteins (e.g., Occludin, VE-cadherin) for maintaining barrier integrity (FIG. 1A, left). During sepsis, inflammatory mediators disturb normal actin dynamics, leading to increased actin fiber bundling and stress fiber formation that generate pulling forces and compromise EC contact stability (FIG. 1A, right). Hence, the modification of actin filament dynamics in ECs would be a valuable approach to the development of treatment options for sepsis. Over the past decade, most studies have focused on two major pathways, i.e., those associated with small GTPases and those associated with myosin light chain (MLC) kinases and phosphatases that control actin dynamics and cell contraction. However, another key signaling pathway involving LIM kinases (LIMK) and their downstream target, Cofilin, has been less well studied in sepsis-induced vascular leak.

At present, there is evidence that Cofilin depolymerizes F-actin to provide new G-actin monomers for polymerization. Cofilin activity is tightly controlled by LIMK1/2, which phosphorylate Cofilin at serine 3, whereby its activity is blocked. In contrast, dephosphorylation by the sling-shot homolog 1 (Ssh1) can reactivate Cofilin, which stimulates the severance and depolymerization of F-actin filaments (FIG. 1A). Interestingly, recent meta-analysis of the genome-wide mRNA expression profile of whole blood collected from 6 cohorts of 450 individuals (n=323 sepsis; n=127 healthy donors) identified both LIMK2 and Ssh1.

Currently, understanding Lcn10 functionality is limited to its potential role as a biomarker for heart failure. However, it has never been investigated whether Lon10 plays any major roles in cardiovascular leakage during sepsis. Our data shows that LPS-caused vascular leak is associated with loss of Lcn10, contributing to myocardial dysfunction and animal mortality. Importantly, we discovered that LPS- and CLP-induced reduction of cardiac Lcn10 occurred only in cardiac endothelial cells (ECs) but not in two other cardiac cell types (myocytes and fibroblasts). Accordingly, in vitro overexpression of Lcn10 in mouse cardiac ECs greatly decreased monolayer leakage upon LPS challenge.

Regarding the putative Lcn10-Ssh1 signaling connection, our RNA sequencing data showed that Ssh1 was the most significantly upregulated gene in Lcn10-overexpressing ECs. Consistently, treatment of ECs with recombinant Lcn10 protein (rLcn10) also resulted in a significant upregulation of Ssh1. Accordingly, the actin-binding protein, Cofilin, was activated in both Lcn10-overexpressing and rLcn10-treated ECs, as evidenced by a great reduction of its phosphorylation levels, leading to less stress fiber generation and more cortical actin formation, compared to respective controls.

Further, our data shows that pre-addition of recombinant Lcn10 protein (rLcn10) to cultured cardiac ECs suppresses stress fiber formation and meanwhile, promote cortical actin generation, resulting in decreased monolayer permeability, compared to control cells upon LPS challenge. rLcn10 can be injected into septic mice to reduce vascular permeability, attenuate cardiac edema and dysfunction, and thus lead to enhanced animal survival. Given that increased vascular leakage is a hallmark of many pathological conditions, such as heart failure, atherosclerosis, and a variety of inflammatory diseases including sepsis, the present invention is likely to have a profound impact on improving human health.

### Lcn10 as a Novel Cofilin Activator

Over the past decade, in order to restore vascular barrier integrity in sepsis, tremendous effort has been spent on how to stabilize endothelial junctions and glycocalyx by controlling the activation of small GTPase signaling, metalloproteases (e.g., ADAMs, MMPs) and transmembrane receptors (e.g., Robo4, Tie2). Far fewer studies have focused on exploring the Ssh1-Cofilin signaling pathway as a means to enhance actin re-organization against vascular leak. Our data has identified Lcn10 as a novel activator of Cofilin in ECs though the upregulation of Ssh1 expression.

FIG. 1B suggests that the net activity of Cofilin might be reduced in blood cells during sepsis. Moreover, Gorovoy et al. recently showed that LINK deficiency in mice greatly suppressed endotoxin-induced vascular leak and improved animal survival. Nonetheless, whether elevation or activation of Ssh1 provides protection against sepsis-triggered vascular leak remains unclear. The present invention has found that both addition of recombinant Lcn10 protein (rLcn10) and forced overexpression of Lcn10 in ECs significantly upregulated Ssh1 expression (see data below). By contrast, siRNA-mediated knockdown of Ssh1 remarkably diminished Lcn10-induced reduction of endothelial permeability upon LPS insult.

The present invention concerns the role of Len10 in vascular permeability during polymicrobial sepsis. Based on available data, we know that: 1) increased vascular leakage is responsible for sepsis-caused hypoperfusion, heart failure and mortality; 2) Lcn10 expression is significantly down-regulated in the blood of sepsis non-survivals, compared to survival patients; and 3) the expression level of Len10 is greatly reduced in human failing hearts and septic mouse hearts (data below). Of interest, our data showed that sepsis-induced reduction of cardiac Lcn10 occurred only in ECs, but not in either fibroblasts or cardiomyocytes. Taken together, these results indicate that Len10 plays a critical role in the regulation of vascular permeability during sepsis. Relevant to this indication, our data revealed that Lcn10-KO mice had a higher vascular leakage, a deteriorated cardiac function, and a lower survival rate, compared to WT mice following LPS insult. By contrast, in vitro overexpression of Lcn10 in ECs showed a greater resistance to LPS-caused monolayer leak than control ECs.

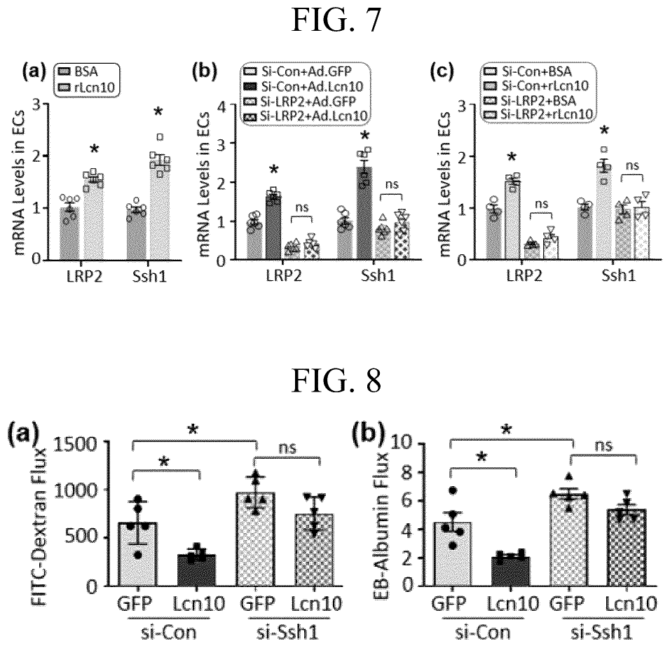

The present invention also involves investigating the therapeutic potential of recombinant Lcn10 protein in treating sepsis. Pre-clinical studies conducted for the present invention found that addition of recombinant Lcn10 protein (rLcn10, 200 ng/ml) to cardiac ECs greatly upregulated Ssh1 expression (FIG. 7A), resulting in enhanced formation of cortical actin and reduced permeability upon LPS exposure (data shown below). These findings indicate that administration of rLcn10 to septic mice will decrease cardiovascular leakage and cardiac dysfunction, and as a consequence, improve animal survival.

### Treatment

In one embodiment, the present invention is a method of reducing the risk of a sepsis-induced vascular leak, tissue edema or organ dysfunction in a subject. The method involves administering an effective amount of a composition selected from the group consisting of Lipocalin 10 (SEQ ID NO: 1), a truncated Lipocalin 10 (Lcn10) protein having the amino acid sequence SEQ ID NO: 2, Lcn10-expressing vectors for full length/truncated Lcn10, or combinations thereof to the subject. In one embodiment, the subject is administered the composition at a dosage of 50-200 ng/g body weight. In another embodiment, the subject is administered with the composition via vein injection.

```
SEQ ID NO: 1
                10 20 30 40
MRQGLLVLAL VLVLVLVLAA GSQVQEWYPR ESHALNWNKF
                50 60 70 80
SGFWYILATA TDAQGFLPAR DKRKLGASVV KVNKVGQLRV
                90 100 110 120
LLAFRRGQGC GRAQPRHPGT SGHLWASLSV KGVKAFHVLS
              130 140 150 160
TDYSYGLVYL RLGRATQNYK NLLLFHRQNV SSFQSLKEFM
              170 180
DACDILGLSK AAVILPKDAS RTHTILP
(Truncated human lipocalin 10 protein
(aa 20-140)
SEQ ID NO: 2
20 30 40 50
A GSQVQEWYPR ESHALNWNKF SGFWYILATA
                60 70 80 90
TDAQGFLPAR DKRKLGASVV KVNKVGQLRV LLAFRRGQGC
              100 110 120 130
GRAQPRHPGT SGHLWASLSV KGVKAFHVLS TDYSYGLVYL
              140
RLGRATQNYK
```

In another embodiment of the present invention, a method of reducing the risk of a heart attack-induced cardiac dysfunction, atherosclerosis, inflammatory bowel disease or diabetes-induced cardiomyopathy in a subject is provided. The method involves administering an effective amount of a composition selected from the group consisting of Lipocalin 10 (SEQ ID NO: 1), a truncated Lipocalin 10 (Lcn10) protein having the amino acid sequence SEQ ID NO: 2, Lcn10-expressing vectors, modified mRNA of full length/truncated Lcn10, or combinations thereof to the subject. In one embodiment, the subject is administered the composition at a dosage of 50-200 ng/g body weight. In another embodiment, the subject is administered with the composition via vein injection.

In another embodiment of the present invention, a pharmaceutical composition comprising a truncated Lipocalin 10 protein having the amino acid sequence SEQ ID NO: 2 is provided. In one embodiment, the composition is for use in a method of reducing the risk of a sepsis-induced vascular leak, tissue edema or organ dysfunction in a subject. In another embodiment, the composition is for use in a method of reducing the risk of a heart attack-induced cardiac dysfunction, atherosclerosis, inflammatory bowel disease or diabetes-induced cardiomyopathy in a subject.

### Lcn10 as Novel Regulator for Correcting the Imbalanced Macrophage Polarization in Diabetic Hearts

Diabetic cardiomyopathy (DCM) is characterized by ventricular dysfunction that may be ascribed to abnormal macrophage function in the heart. How to modulate macrophage activity in diabetic hearts remains elusive. Recent meta-analysis data reveal that lipocalin 10 (Lcn10) is significantly downregulated in cardiac tissue of patients with heart failure. However, the functional roles of Lcn10 in macrophages under diabetic condition has never been explored.

A study was conducted to examine the role of Lcn10 in DCM (see Example 26). The data shows that Lcn10 plays an essential role in modulating macrophage phenotypic change under stress conditions. Loss of Lcn10 aggravates pro-inflammatory phenotypes in macrophages through damping of Nr4al pathway, leading to impaired cardiac function in diabetic mouse model.

### Lcn10 as a Novel Protector Against Sepsis-Induced Cardiovascular Leakage

Despite recent advances in antibiotic therapy and supportive critical care, sepsis remains a leading cause of death in intensive care units. The increased vascular leakage seen in sepsis patients is well-recognized to be responsible for sepsis-triggered organ failure and patient mortality. Recently, we and others have shown a positive correlation between cardiovascular permeability, cardiac depression and mortality during sepsis. However, the mechanisms underlying sepsis-induced cardiovascular leakage remain obscure. Prior work suggests that lipocalin 10 (Lcn10), a member of the lipocalin superfamily, is significantly downregulated in the blood of non-survival septic patients when comparing to the survival group. Nonetheless, whether circulating Lcn10 affects endothelial barrier integrity in sepsis remains unknown.

A study was conducted to examine the role of Lcn10 in sepsis-induced cardiovascular leakage (Example 27). The results show that endothelial Lcn10 is critical for protecting against sepsis-induced cardiovascular leakage via the activation of the Ssh1-Cofilin pathway. The study results indicate that Lcn10 could be a novel regulator of vascular barrier integrity and as a new protector against sepsis-induced organ failure.

### Len 10 as a Novel Regulator of Macrophage Efferocytosis in Ischemic/Reperfused Hearts

Efficient clearance of dead cells by macrophages (termed as efferocytosis) is critical for timely repairing the injured heart after ischemia/reperfusion (I/R), as defective removal of dying cells could cause secondary necrosis and infarct expansion. Recent studies have shown that lipocalin 10 (Lcn10) is significantly downregulated in cardiac tissue from patients with heart failure. However, the role of Len10 is virtually unknown during cardiac I/R, and its function in macrophage efferocytosis has never been explored. Our initial data showed that Len10 expression was upregulated in bone marrow-derived macrophages (BMDMs) during efferocytosis. Thus, we hypothesized that knockout (KO) of Lcn10 would diminish macrophage efferocytosis, leading to exacerbated cardiac I/R injury.

A study was conducted to examine the role of Len10 in macrophage efferocytosis (Example 28). Our data indicate that Len10 is pivotal for macrophage efferocytosis to remove cardiac dead cells during I/R. Thus, Lcn10 could be used as a new mediator for the treatment of cardiac I/R injury.

### EXAMPLES

### Genetic Mouse Models

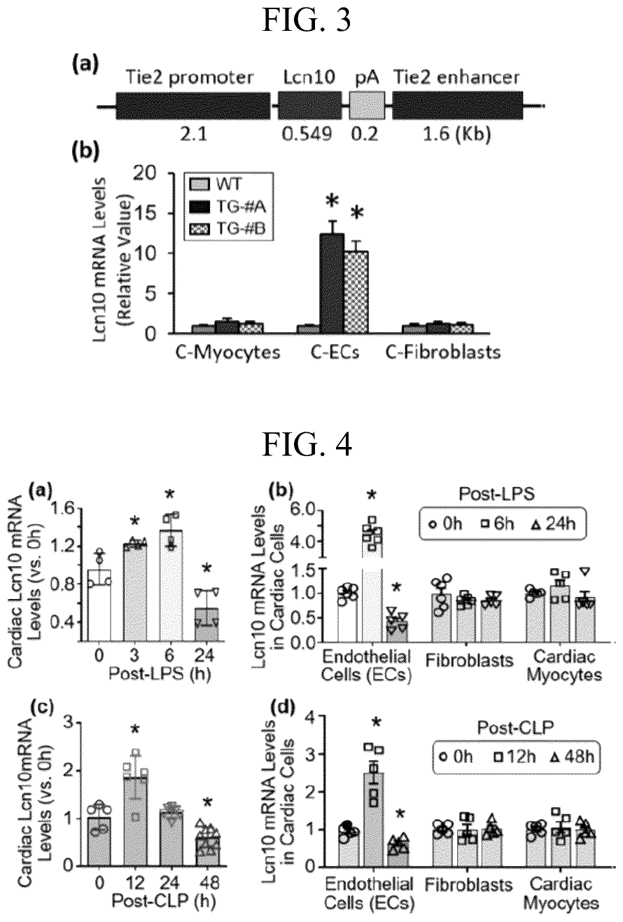

A global Lcn10-knockout (KO) mouse model was purchased from UC-Davis KOMP Repository (Lcn10tm1.1) (FIG. 2). Two novel transgenic mouse lines (EC-Lcn10-Tg, #A and #B) were also generated in which Lcn10 is overexpressed specifically in endothelial cells (ECs), using an EC-specific Tie2 promoter (FIGS. 3A and 3B). Ssh1-KO mouse model was acquired and was bred with EC-Lcn10-Tg mice to generate a new mouse model (EC-Lcn10Tg/Ssh1−/−) in which Ssh1 is ablated and Len10 is overexpressed in ECs. The three genetic mouse models (Lcn10-KO, EC-Lcn10-Tg, and EC-Lcn10Tg/Ssh1−/−) are on the C57BL/6 background. All animal procedures have been approved by our Institutional Animal Care and Use Committee.

### Example 1—Expression Levels of Lcn10 are Altered Upon LPS Treatment and CLP Surgery

Lcn10 was initially identified as an epididymis-specific gene. Later RNA-seq. analysis showed that Lcn10 is ubiquitously expressed and enriched in the blood, spleen and heart. While prior studies reported that the Lon10 expression in blood cells was significantly lower in non-survival sepsis patients than survivals, it remains unclear whether cardiac Lcn10 expression is altered under sepsis conditions. To test this, we first measured expression levels of cardiac Lcn10 in mice (2-month old) after LPS injection (10 μg/g). RT-qPCR results showed that Lcn10 expression was greatly increased in mouse hearts at 3 and 6 h after LPS treatment, but dramatically reduced at 24 h post-LPS injection (FIG. 4A). Next, endothelial cells (ECs), fibroblasts, and myocytes were isolated from these LPS-treated hearts to further assess Lcn10-mRNA levels. Interestingly, we observed that LPS-caused alterations of cardiac Len10 occurred only in ECs but not in fibroblasts or myocytes (FIG. 4B). Similarly, cardiac Lcn10 expression was remarkably upregulated in mice (2-month old) at 12 h post-CLP surgery, whereas it was significantly downregulated at 48 h post-CLP (FIG. 4C). Consistently, such CLP-induced up-/down-regulations of cardiac Lcn10 expression occurred only in ECs but not in either fibroblasts or cardiomyocytes (FIG. 4D). Taken together, these data indicate that Lcn10 is involved in the regulation of vascular permeability during sepsis. Its reduction at the late phase of sepsis may directly contribute to sepsis-induced cardiovascular leak, whereas its elevation at the early phase of sepsis could act as a compensatory mechanism.

### Example 2—Knockdown of Lcn10 in Cardiac ECs Increases LPS-Triggered Permeability

To test whether reduced Lcn10 expression in ECs contributes to sepsis-induced vascular leak, we next transfected mouse cardiac ECs (MCECs, purchased from CELLutions Biosystems Inc) with Lcn10-siRNA (siLcn10) or control siRNA (SiCon) for 48 h. Then, these cells were harvested and seeded onto the upper chamber of a transwell system until a monolayer was formed, followed by addition of LPS (1 μg/ml) to induce EC leak. Using three different methods [trans-endothelial electrical resistance (TEER), FITC-labeled dextran and Evans blue dye (EB)-albumin flux assays], we observed that addition of LPS to siRNA-control cells caused remarkable drops in TEER values, which were further exaggerated in siLcn10-transfected cells. Consistently, LPS-induced flux of FITC-dextran and EB-bound albumin was significantly increased in siLcn10-cells, compared to siRNA-control cells. These results indicate that down-regulation of Lcn10 can promote endothelial permeability during sepsis.

### Example 3—Lcn10-Null Mice Exhibit the Increased Cardiovascular Leak Upon LPS Exposure

To evaluate whether Lcn10-deficiency affects vascular permeability in vivo during sepsis, an endotoxemia model was utilized by intraperitoneal (i.p.) injection of LPS (10 μg/g body weight) for 24 h, followed by the tail vein injection of 1% Evans blue dye (200 μl). The Lcn10-KO mice used for the study were healthy and did not show any obvious pathological abnormalities. Upon LPS challenge, however, these Lcn10-KO mice showed severe vascular leak, as evidenced by: 1) increased extravasation of EB dye in the aorta and the heart, edema was further confirmed by the ratio of wet/dry weight), and 2) increased release of EB dye to the extravascular compartment of frozen aorta sections and heart sections (note: EB is red under a fluorescence microscope, compared to LPS-treated WT controls. Therefore, these results show that knockout of Lcn10 promotes sepsis-induced cardiovascular leak and tissue edema.

### Example 4—Lcn10-KO Exaggerates Cardiac Depression and Mortality in LPS-Treated Mice

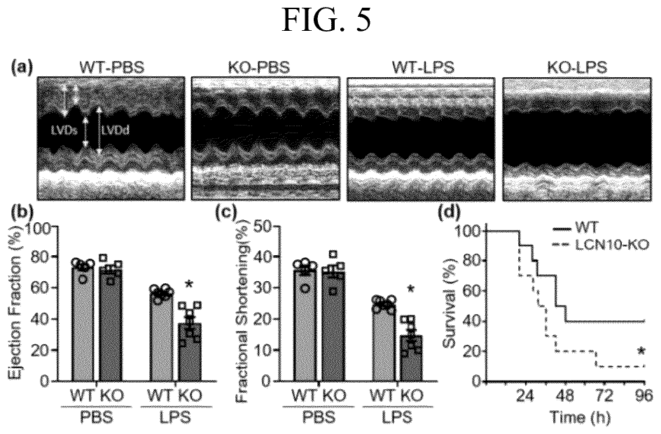

A test was conducted to determine whether increased vascular permeability in LPS-treated Lcn10 mice could affect cardiac function and animal survival. Echocardiography analysis (FIGS. 5A, 5B and 5C) showed that absence of Lcn10 did not affect cardiac function under basal PBS conditions, but it did exaggerate LPS-induced myocardial depression, as evidenced by significant decreases in the left ventricular ejection fraction (EF %) and fractional shortening (FS %), compared to LPS-treated WTs (p<0.05). In addition, loss of Lcn10 had major impact on mortality during endotoxemia. Nine out of 10 KO mice (90%) died at 4 days post-LPS injection, whereas 40% of WT mice (n=10) remained alive (FIG. 5D, p<0.05). Collectively, these data indicate that Lcn10-deficiency contributes significantly to LPS-triggered myocardial dysfunction and lethality.

### Example 5—Overexpression of Len10 in Cardiac ECs Decreases Permeability Upon LPS Treatment

The data above strongly indicate that reduction of Lcn10 aggravates LPS-induced vascular leakage, cardiac depression and mortality. However, it remains unclear whether forced overexpression of Lcn10 in ECs attenuates vascular leakage during sepsis. To this end, we performed studies using cultured mouse cardiac ECs (MCECs), which were infected with recombinant adenovirus vector Ad.Lcn10 expressing both Lcn10 and GFP for 48 h. MCECs infected with Ad.GFP encoding GFP alone were used as controls. Overexpression of Lcn10 in Ad.Lcn10-ECs was validated by RT-qPCR. In parallel, these Ad-infected cells were harvested and seeded onto the upper chamber of a transwell system until an EC monolayer was formed. Then, LPS (lug/ml) was added, followed by measurement of EC leakage as described above. We observed that LPS-caused a drop of TEER in Ad.GFP-cells, which was greatly suppressed in Ad.Lcn10-cells. Similarly, the flux analysis of FITC-dextran and EB-albumin both showed that Ad.Lcn10-cells had a lower permeability than Ad.GFP-cells upon LPS challenge. Collectively, these findings indicate that the elevation of Lcn10 in ECs could decrease endotoxin-triggered vascular leakage.

### Example 6—RNA-Seq Analysis

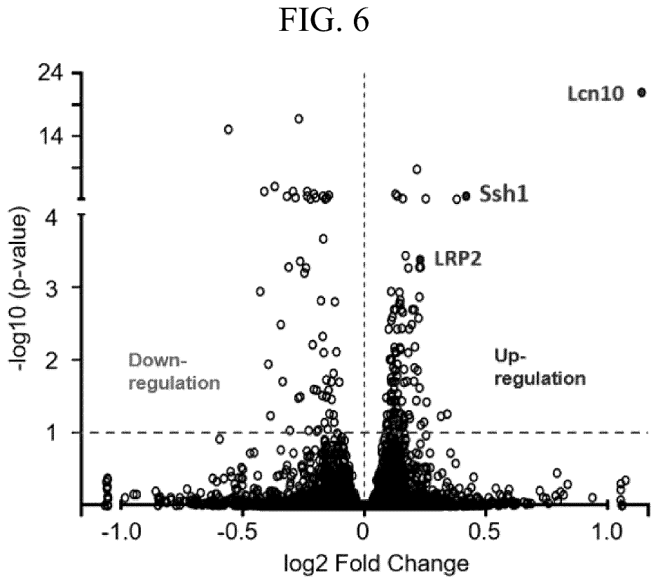

Our RNA-seq analysis revealed that a total of 147 genes were differentially expressed including 102 up-regulated and 45 down-regulated in Ad.Lcn10-ECs, compared to Ad.GFP-cells (FIG. 6). Furthermore, gene ontology enrichment analysis results showed that elevation of Lcn10 in ECs greatly activated two major signaling pathways involved in the regulation of actin cytoskeleton and cell junction, which were confirmed with RT-qPCR (data not shown). Of interest, Ssh1 is the most significantly upregulated gene in Len10-cells (FIG. 6). Prior work has indicated that Ssh1 is a phosphatase which activates Cofilin, a key actin-binding protein that plays an essential role in modulating actin filament dynamics. Indeed, our latest data (below) revealed that overexpression of Lcn10 in ECs yielded less stress fibers and more cortical actin filaments than control cells upon LPS insult. Accordingly, knockdown of Ssh1 in Lcn10-ECs by siRNAs was found to greatly suppress Lcn10-elicted effects on actin filament turnover, leading to increased permeability, compared to control cells following LPS insult. Hence, these data indicate that Lcn10-elicited inhibition of vascular leakage during sepsis acts through Ssh1-mediated actin filament re-organization.

### Example 7—Lcn10 Regulates Ssh1 Expression in ECs Via LRP2

To identify possible mediators for the Lcn10-induced upregulation of Ssh1 in ECs, we first analyzed the expression data for membrane receptors since Len10 is a secreted protein. Available data shows that lipocalin family members interact with LDL receptor-related proteins (LRPs). As implicated here, our RNA-seq data revealed that the expression of LRP2 was significantly elevated in Lcn10-cells compared to control GFP-cells (FIG. 6). This indicates a positive feedback loop involving Lcn10. Consistently, addition of recombinant Lcn10 protein (rLcn10, 200 ng/ml) to cultured ECs promoted expression of both LRP2 and Ssh1, compared to BSA-controls (FIG. 7A). Furthermore, when LRP2 was knocked down by siRNA (si-LRP2), the Ssh1 expression was not up-regulated in either Ad.Lcn10-infected (FIG. 7B) or rLcn10-treated ECs (FIG. 7C). These data indicate that Len10 upregulates Ssh1 expression in ECs through its interaction with LRP2.

### Example 8—LPS Injection and CLP Surgery Both Result in Downregulation of Ssh1 in Cardiac ECs

Next, to determine whether sepsis conditions affect Ssh1 expression in ECs, we performed RT-qPCR, and observed that expression levels of Ssh1 were greatly reduced in cardiac ECs of mice at 24h post-LPS injection, compared to PBS-controls. Likewise, expression levels of LRP2, as an upstream regulator of Ssh1, were remarkably lower in LPS-treated cardiac ECs than in control cells. Consequently, VE-cadherin levels on the surface of LPS-treated cardiac ECs were significantly decreased, compared to PBS-controls, as measured by flow cytometry analysis. Similar findings were observed in cardiac ECs isolated from mice at 48h post-CLP. These data correlate well with the reduced expression of Len10 in cardiac ECs of mice upon LPS-injection and CLP surgery (FIG. 4), indicating that sepsis-induced vascular leakage is associated with a reduction in Lcn10-LRP2-Ssh1 signaling.

### Example 9—Overexpression of Lcn10 in ECs

Available data has shown that Ssh1 activates Cofilin by dephosphorylation at Ser-3. Accordingly, we measured Cofilin and its Ser-3-phos-phorylated levels in Ad.Lcn10-infected ECs and control cells by Western-blotting. We observed that Ad.Lcn10-ECs displayed higher levels of Ssh1 than Ad.GFP-cells, consistent with our RNA-seq data above. The phosphorylated levels of Cofilin were significantly lower in Ad.Lcn10-cells than in Ad.GFP-cells, suggesting its dephosphorylation and activation by Ssh1. Given that Cofilin is well known to control actin filament reorganization by stimulating the severance and depolymerization of actin filaments, we next examined cytoskeletal structures (i.e., stress fibers and cortical actin networks), using Alexa-Fluor 594 phalloidin (F-actin probe) staining. Under basal conditions, GFP-ECs showed a cortical organization of cytoskeletal F-actin along the cell periphery. After LPS stimulation, peripherally located F-actin is substituted by centralized and irregular stress fibers running across the cytoplasm. However, overexpression of Lcn10 strongly reinforced peripheral actin bands, both in unstimulated and LPS-treated cells. Importantly, LPS-triggered formation of stress fibers was less pronounced in Lcn10-cells than in control GFP-cells. Given that reorganization of the actin cytoskeleton affects the stability of EC junctions and paracellular gap formation, we lastly evaluated the integrity of cell peripheral membrane by staining these ECs with antibody to ZO-1, a tight junction protein. The linear shape of cell-cell junctions was displayed in both GFP- and Lcn10-cells under basal conditions. However, LPS treatment greatly impaired the integrity of cell membranes, as evidenced by jagged and disconnected ZO-1 staining in GFP-cells, but not in Lcn10-cells. Together, these data indicate that forced elevation of Lcn10 in ECs activates the Ssh1-Cofilin pathway and reinforces the cortical actin network upon LPS insult, leading to reduced stress fiber formation and opening of cell junctions.

### Example 10—Knockdown of Ssh1 in ECs

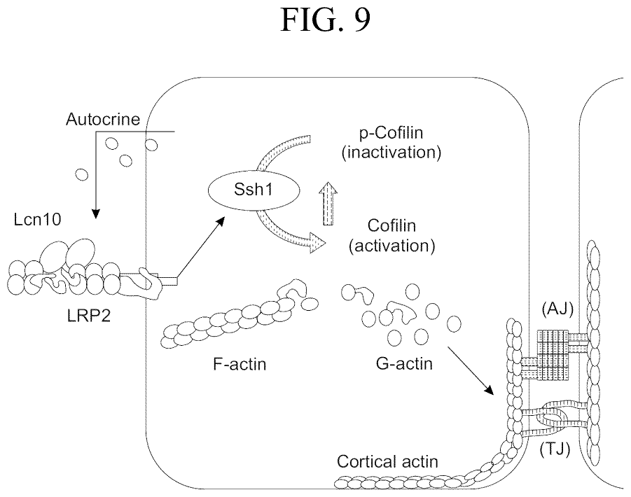

To test whether Lcn10-elicited action is dependent on Ssh1 signaling, we first utilized sequence-specific siRNAs to knockdown Ssh1 expression in Ad.Lcn10-infected ECs, followed by LPS challenge for EC permeability analysis. Western-blotting data revealed that elevation of Ssh1 in Ad.Lcn10-ECs was greatly reduced by siRNA-Ssh1 to a similar level as in GFP-cells. Consequently, Lcn10-induced decrease in the levels of phosphorylated Cofilin was greatly blocked by siRNA-Ssh1 transfection, as revealed by similar degree of Cofilin phosphorylation between siRNA-Ssh1/Ad.Lcn10-ECs and control siRNA-Ssh1/Ad.GFP-cells. EC permeability analysis further indicated that FITC-dextran flux was greatly reduced in Lon10-cells, compared to GFP-cells following LPS challenge (FIG. 8A). However, such LPS-caused leakage was significantly increased in Lcn10-cells when Ssh1 was knocked down by siRNAs (FIG. 8A). Similar findings were observed using the EB-albumin flux assay (FIG. 8B). Taken together, these initial mechanistic data indicate that Lcn10-mediated effects in LPS-triggered EC leakage may be largely dependent on the Ssh1 signaling. Therefore, these data show that elevation of Lcn10 in ECs increases Ssh1 expression through autocrine mechanisms via the LRP2 receptor, leading to dephosphorylation of Cofilin (activation, FIG. 9). Consequently, the formation of cortical actin filaments is enhanced, and stress fiber formation is suppressed upon LPS insult (FIG. 9). Importantly, knockdown of Ssh1 in ECs by siRNAs significantly limits the Lcn10-elicited reduction of monolayer leakage. Without being bound by theory, we hypothesize that the mechanism underlying Lcn10-induced protection against cardiovascular leak during sepsis is dependent on Ssh1-mediated actin filament re-arrangement.

### Example 11—rLcn10 Inhibits Stress Fiber Formation and Decreases Leakage in LPS-Treated ECs

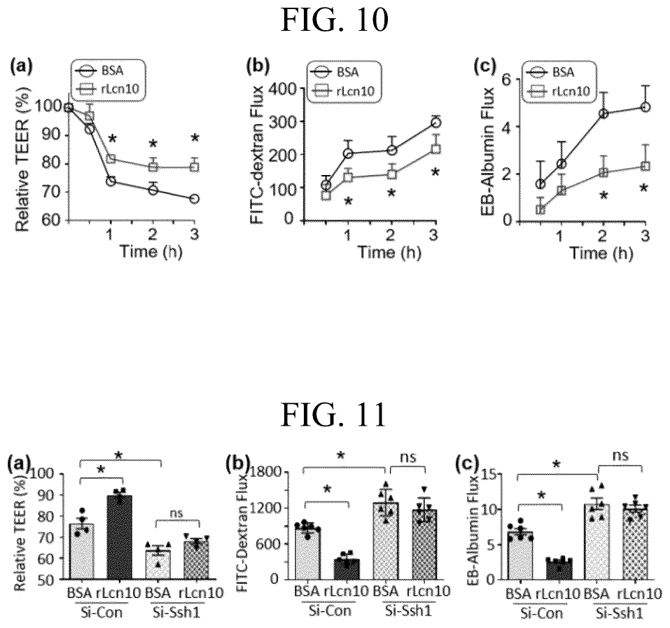

To address whether exogenous Lcn10 protein has therapeutic effects against sepsis-triggered vascular leak, we conducted studies using cardiac ECs, and treated these cells with rLcn10 protein (purchased from MyBioSource Inc.) or control BSA for 12h, followed by the addition of LPS (1 μg/ml) for barrier integrity and permeability assays. (Note: endotoxin levels in rLcn10 and BSA proteins are <0.01EU/μg, low enough to exclude its effects). We observed that treatment with rLcn10a significantly increased trans-endothelial electrical resistance (TEER) in a time-dependent manner, compared to control BSA-treated cells after LPS insult (FIG. 10A). Accordingly, the leakage of FITC-dextran (FIG. 10B) and EB-albumin (FIG. 10C) was greatly inhibited in rLcn10-ECs, compared to control cells following LPS challenge. In follow-up studies, we selected the time point of 2 h post-LPS treatment for actin filament analysis and stained these ECs with Alexa Fluor 488 phalloidin. In control BSA-cells after LPS stimulation, peripherally located F-actin filament was re-organized into stress fiber bundles, spreading across the cytoplasm. However, treatment with rLcn10 strongly reinforced peripheral actin (cortical actin) bands and inhibited stress-fiber formation upon LPS insult. Together, these data indicate that exogenous addition of rLcn10 promotes the assembly of cortical actin filaments and counters stress fiber formation, thereby inhibiting LPS-triggered vascular leak.

### Example 12—rLcn10-Mediated Protective Effects Against LPS-Caused EC Leakage

Given that rLcn10 upregulates endothelial Ssh1 expression, we next tested whether rLcn10-elicited decrease of EC monolayer leakage is dependent on Ssh1. ECs were transfected with siRNA-Ssh1 (Si-Ssh1) or siRNA-control (Si-Con) for 48 h, followed by addition of rLcn10 (200 ng/ml) or BSA control. Then, EC leakage was measured at 2 h post-LPS challenge. We observed that rLcn10-induced reduction of EC permeability was significantly blocked by siRNA-Ssh1 transfection, as evidenced by similar degrees of TEER and leakage of FITC-dextran and EB dye between BSA- and rLcn10-treated cells (FIGS. 11A, 11B and 11C). These data indicate that Ssh1 plays an essential role in Len10-induced benefits in reducing vascular leakage during sepsis.

### Example 13—rLcn10 Decreases LPS-Induced Cardiovascular Leak In Vivo

To test whether rLcn10 has therapeutic effect in reducing vascular leakage during sepsis, the following studies were conducted: 8-week old mice were received rLcn10 (200 ng/g) or BSA control via the tail veil injection at 1h post-LPS treatment. 24h later, EB dye was injected intravenously for the analysis of cardiovascular permeability, as described previously. We observed that treatment of LPS-mice with rLcn10 reduced EB extravasation to a greater degree in the heart and aorta, compared to BSA-treated controls. These data indicate that rLcn10 can decrease cardiovascular leak during sepsis.

### Example 14—the Expression Levels of Lcn10 in Tissues of Septic Mice

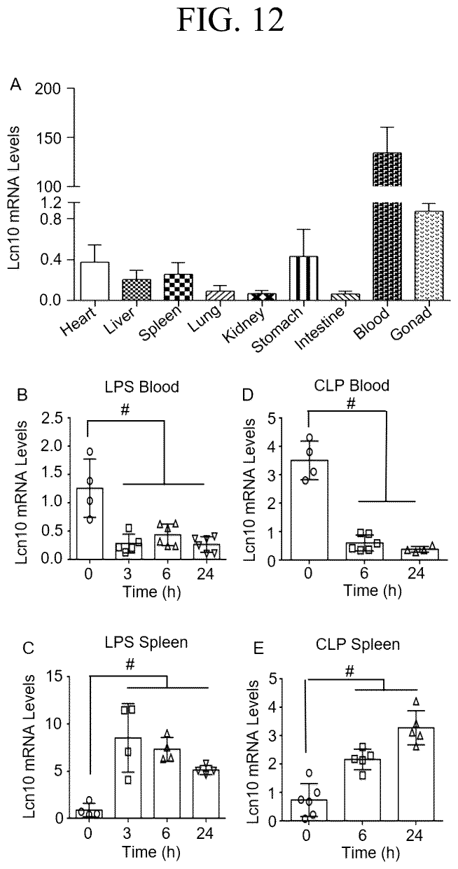

mRNA levels of Lcn10 in different tissues of C57/6J mice were analyzed by RT-qPCR (FIG. 12A). After intraperitoneal injection of LPS (10 mg/kg), mice were sacrificed at 0, 3, 6 and 24 h, and the mRNA levels of Lcn10 in the blood (FIG. 12B) and the spleen (FIG. 12C) were assessed. At the time points of 0, 6 and 24 h after CLP-operation in mice, mRNA levels of Lcn10 in the blood (FIG. 12D) and the spleen (FIG. 12E) were determined by RT-qPCR. n=4-6 for each group at the indicated time. #, P<0.05.

### Example 15—Lcn10 Deficiency Aggravates CLP-Induced Multiple Organ Injury

Western-blot was used to detect Lcn10 expression in the spleens of WT and Lcn10 KO mice. GAPDH was used as a loading control. Kaplan-Meier survival curves and the log-rank (Mantel-Cox) test were used to detect survival rate. Lcn10-KO mice showed a lower survival rate than WT mice within 96 hours after CLP operation. At 24 h post-CLP surgery, WT and Lcn10-KO mice were sacrificed for collecting the livers and kidneys, and then stained with hematoxylin and eosin. Representative images of the liver and kidney were shown. Scale bars, 20 μm. At 16 h after CLP operation, levels of ALT and Cr in the serum in WT and Lcn10-KO mice were analyzed by ELISA. #, P<0.05; n=4-6 mice per group.

### Example 16—Lcn10 Deficiency Increases Inflammatory Response

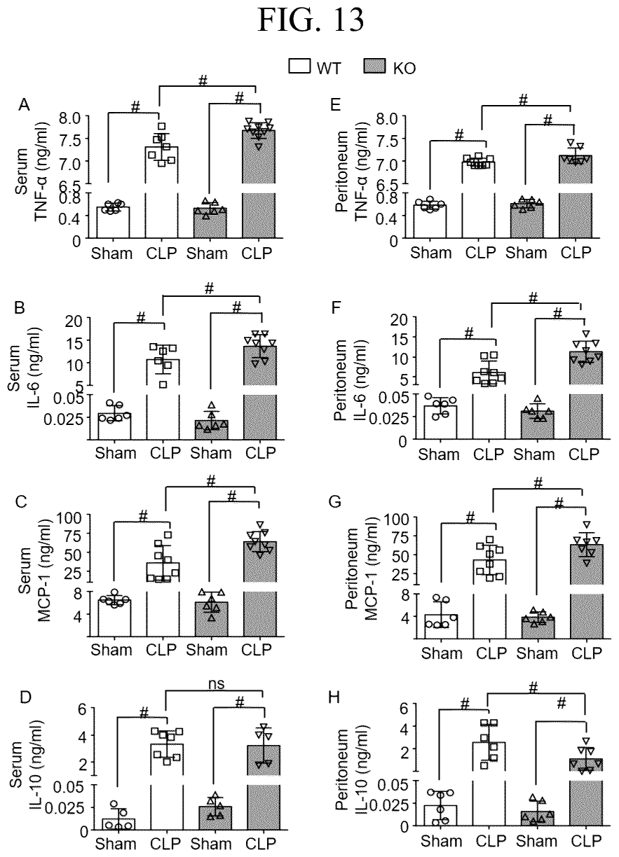

Serum and peritoneal lavage fluid were harvested at 16 h after CLP-operation (FIG. 13). The pro-inflammatory cytokines TNF-α (FIG. 13A, FIG. 13E), IL-6 (FIG. 13B, FIG. 13F) and MCP-1 (FIG. 13C, FIG. 13G) and anti-inflammatory cytokine IL-10 (FIG. 13D, FIG. 13H) were measured using ELISA kits. #, P<0.05; n=6-8 mice per group. The data shows that Lcn10 deficiency increases inflammatory response locally and systemically in sepsis.

### Example 17—Lcn10 Deficiency Attenuates Bacterial Clearance and Suppressed Phagocytic Capability of Peritoneal Macrophages (PMs) from Septic Mice

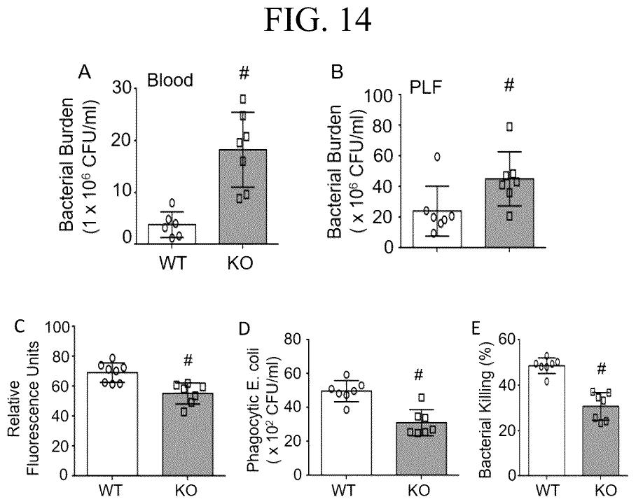

The bacterial burden in both blood (FIG. 14A) and peritoneal lavage fluid (PLF, FIG. 14B) in Lcn10-KO mice were compared with WT mice after CLP operation. (FIG. 14 C) The PMs obtained from WT and Lcn10-KO CLP mice were incubated with the fluorescent labeled E. coli particles for 1.5 h and then examined under confocal microscopy for phagocytosis analysis by measuring the mean fluorescence intensity (FIG. 14C). In addition, the entry of living E. coli by PMs was determined by bacterial CFU counts after incubating with living E. coli for 1.5 h (FIG. 14D). The ratio of CFU at 4 h to CFU at 1.5 h after incubating with living E. coli was assessed, and the percentage of bacterial killing was calculated (FIG. 14F). #, P<0.05, n=6-8 per group.

### Example 18—Dynamic Expression of Lcn10 in Septic Models

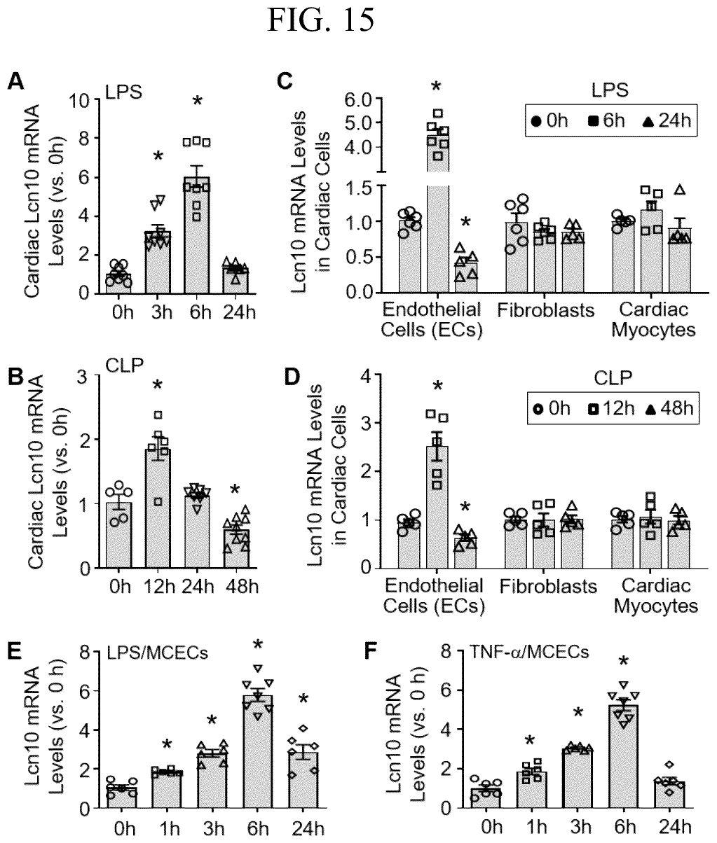

At Oh, 3 h, 6 h and 24 h after intraperitoneal injection of LPS (10 mg/kg BW), mouse hearts were harvested for mRNA measurement by RT-qPCR (n=6-8). Hearts were harvested at Oh, 12h, and 48h after CLP and Lcn10 mRNA levels were determined by RT-qPCR (n=5-9). (FIG. 15C, FIG. 15D) Endothelial cells, cardiomyocytes, and fibroblasts were respectively isolated from hearts harvested at appropriate time points both in LPS (0 h, 6 h and 24 h) (FIG. 15A) and CLP (Oh, 12 h and 48 h) (FIG. 15B) models, then total RNAs were collected and assessed by RT-qPCR (n-5-6). (FIG. 15E, FIG. 15F) mRNA expression levels of LCN10 in MCECs (mouse cardiac endothelial cells) at 1 h, 3h, 6 h and 24 h after LPS (lug/ml) (FIG. 15E) and TNF-α (10 ng/ml) (FIG. 15F) stimulation, 18s gene expression was used as the internal control (n-6-7). All results are presented as mean±SEM and analyzed by student's t test. (*, p<0.05).

### Example 19—Knockdown of Lcn10 Augments the Permeability in Mouse Cardiac Endothelial Cells (MCECs)

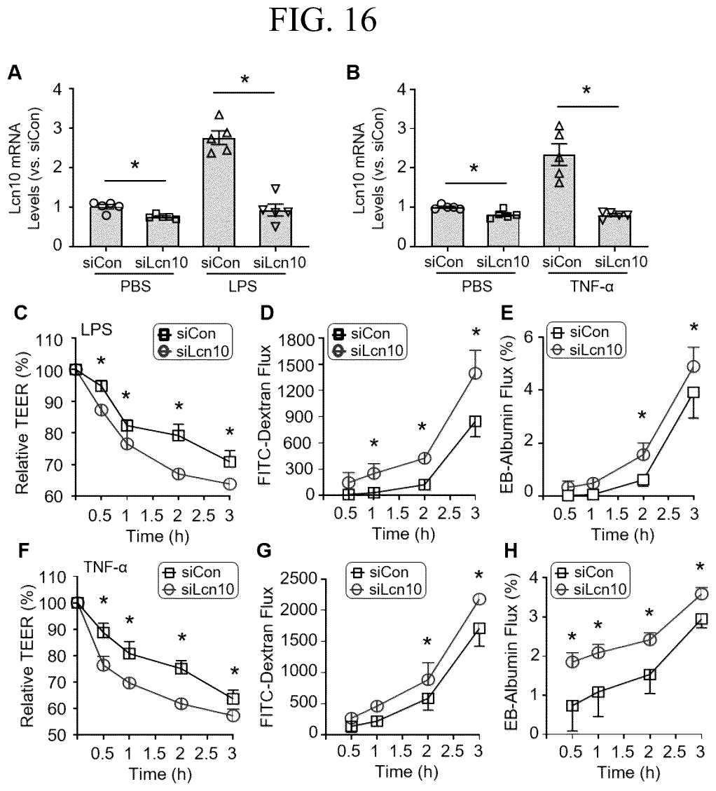

MCECs were transfected with siRNA-Lcn10 (siLcn10) and negative control siRNA (siCon) was used as control. 60h later, cultured cells were treated with PBS, LPS (1 μg/ml) or TNF-α (10 ng/ml) for 3 h. Then the resulting MCECs were harvested to isolate total RNA. (FIG. 16A, FIG. 16B) mRNA levels of Lcn10 were measured by RT-qPCR (n=5). Results are presented as mean±SEM and analyzed by student's t test. (*, p<0.05). (FIG. 16C-FIG. 16E) Compared to negative control group, siLcn10-transfected MCECs displayed significantly increased leakage after LPS treatment, as assessed by three measures: trans-endothelial electrical resistance (TEER) (FIG. 16C) (n=3), FITC-dextran (FIG. 16D) (n=6), and EB-BSA flux (FIG. 16E) (n=6). (FIG. 16F, FIG. 16H) Similar results were observed after treatment with TNF-α including decreased TEER (FIG. 16F) (n=3), increased FITC-dextran (FIG. 16G) (n=6) and EB-BSA flux (H) (n=6). Similar results were obtained in other two independent experiments. Results are presented as mean±SD and analyzed by two-way ANOVA (*, p<0.05).

### Example 20—Lcn10 Deficiency Aggravates Vascular Permeability, Organ Injury and Animal Mortality in Septic Mice

Genotyping of Lcn10+/+ (+/+), Lcn10+/−(+/−), and Lcn10−/−(−/−) mice. Lcn10-KO mice were confirmed by Western blot using heart tissue. Lcn10-KO and WT mice were received LPS injection (10 mg/kg BW, i.p.). Twenty hours later, Evans blue dye (4 mg/kg BW) was intravenously injected into these mice. KO mice displayed a worse peripheral circulation and increased vascular leakage in the heart and aorta. Vascular leakage within the heart and aorta was further quantitative analyzed by incubating these organs with formamide at 55° C. for 48 h, then, the elution was measured the optical density at 620 nm on a spectrophotometer (n=4). EB dye in the extravascular compartment of frozen sections of heart and aorta was examined under a confocal LSM 710 microscope. The intensity of red fluorescence within the heart and aorta was quantified with Image J software (n=5). 20 h after LPS or PBS injection, the cardiac function of LCN10-KO and WT mice was determined by echocardiography. Representative M-mode echocardiography recordings for cardiac function measurement in WT and KO mice after LPS treatment. Left ventricular ejection fraction (EF %) (P) and fractional shortening (FS %) were calculated (n=5-7). (*, p<0.05). Kaplan-Meier survival curves were generated to compare mortality between 2 groups, significance was determined by log-rank (Mantel-Cox) test. (*, p<0.05; n=20 per group).

### Example 21—Overexpression of Full-Length Lcn10 Reduces LPS-Caused Endothelial Cell Leakage

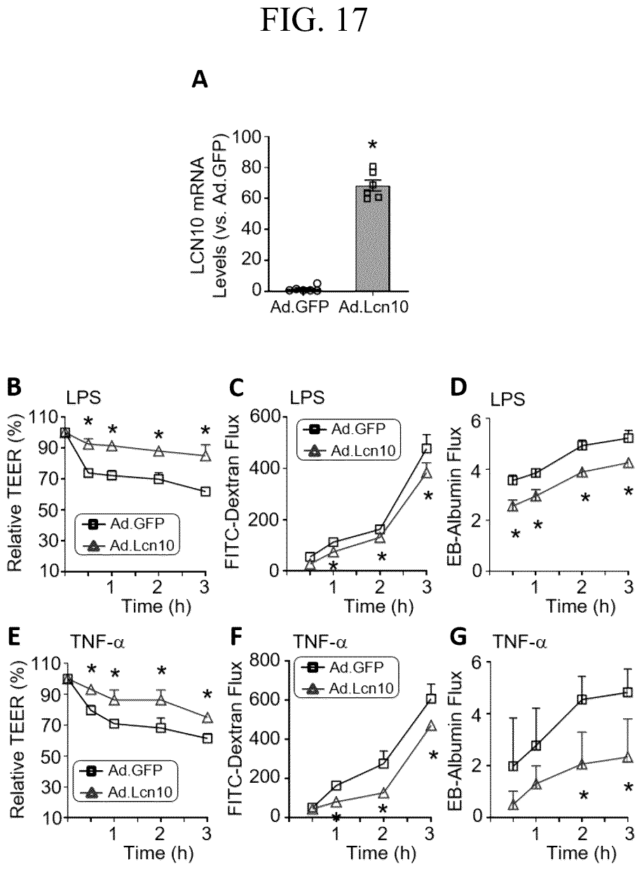

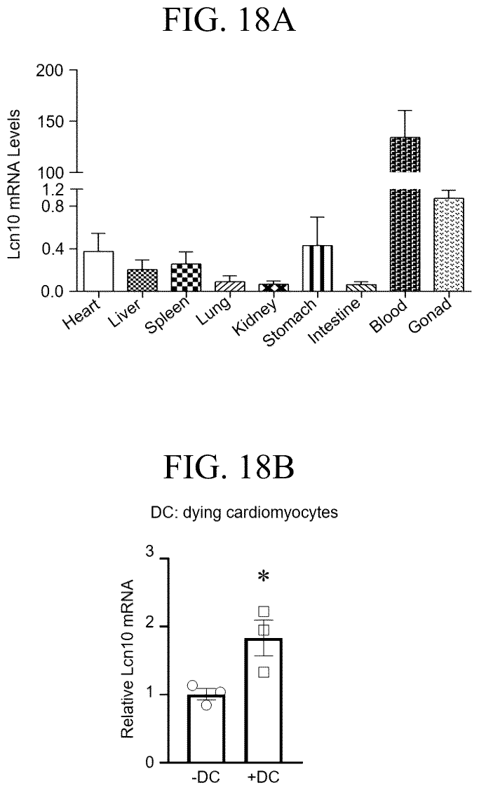

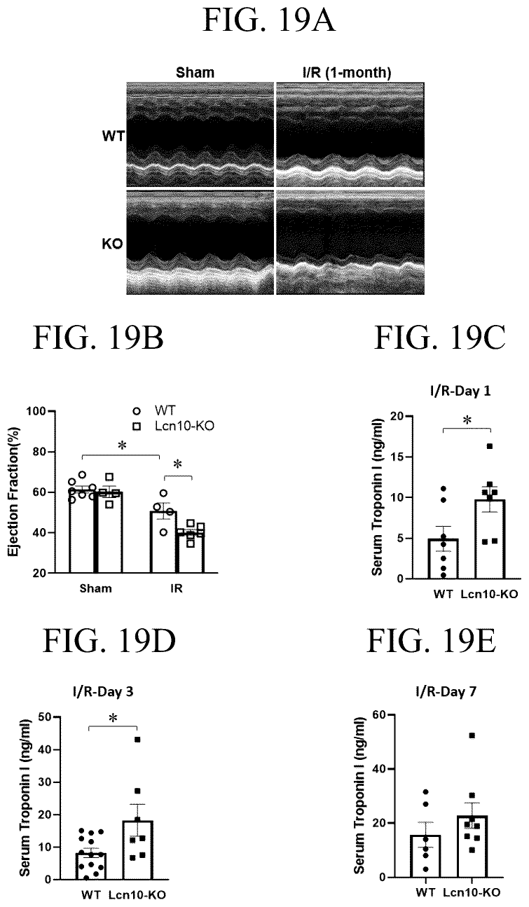

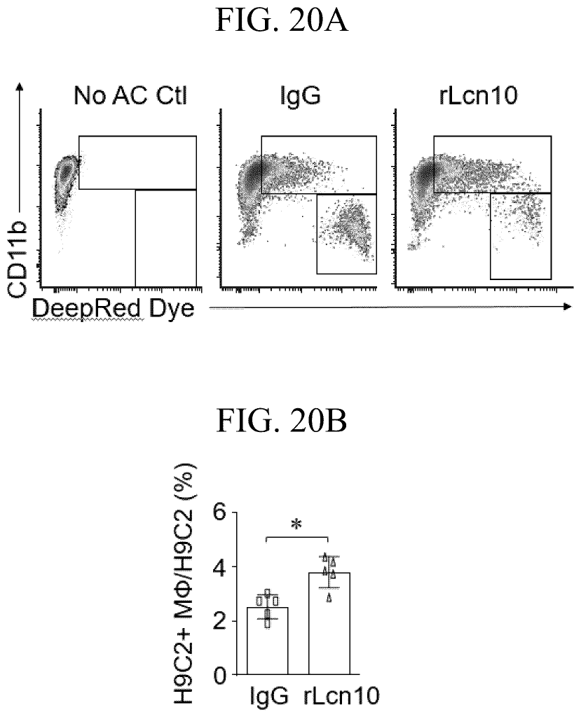

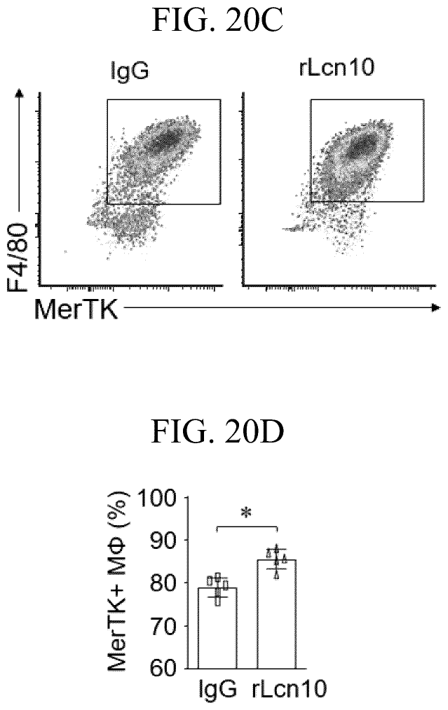

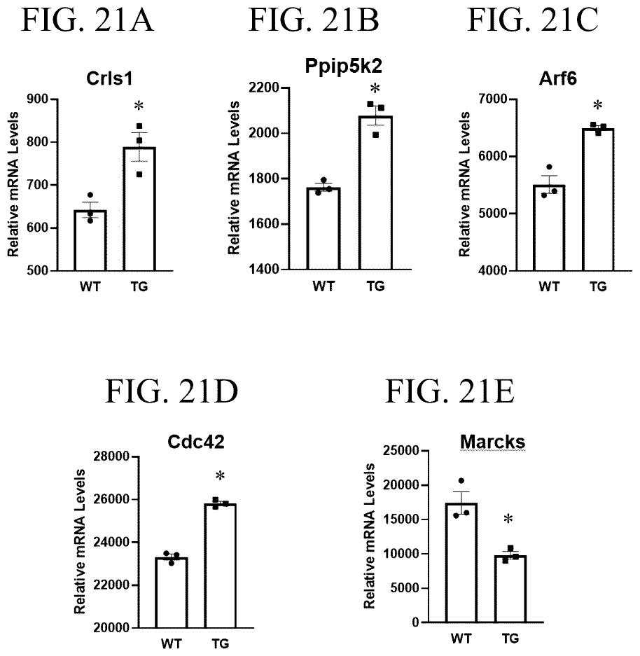

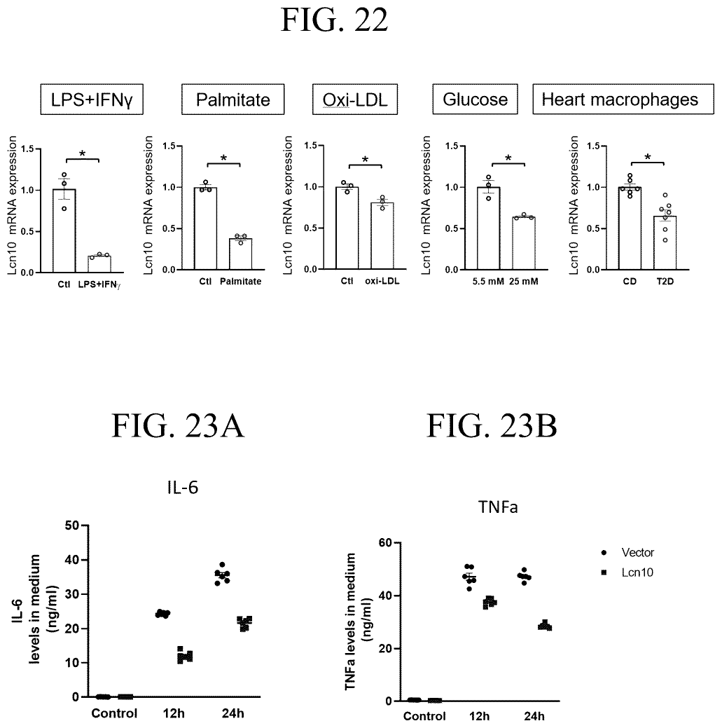

Mouse cardiac ECs (MCECs) were infected with recombinant adenovirus vector Ad.Lcn10 expressing both full-length Lcn10 and GFP for 48 h. MCECs infected with Ad. GFP encoding GFP alone were used as controls. (FIG. 17A) Overexpression of full-length Lcn10 in Ad.Lcn10-ECs was validated by RT-qPCR. (*, p<0.05, n=6). (FIG. 17B-FIG. 17D) At 48 h post-transfection, LPS (1 μg/ml) was added and the permeability of MCECs was measured by three methods: TEER (FIG. 17B), FITC-dextran leakage (FIG. 17C) and EB-BSA flux (FIG. 17D). LPS-caused a drop of TEER in Ad.GFP-cells, which was greatly suppressed in Ad.Lcn10-cells (FIG. 17B). Similarly, the flux analysis of FITC-dextran (FIG. 17C) and EB-albumin (FIG. 17D) both showed that Ad.Lcn10-cells had a lower permeability than Ad.GFP-cells upon LPS challenge. Likewise, higher TEER (FIG. 17E), lower FITC-dextran (FIG. 17F) and EB-BSA flux (FIG. 17G) were observed in Ad. LCN10-transfected MCECs upon TNF-α challenge, compared to controls (*, p<0.05). Collectively, these findings indicate that elevation of Lcn10 in ECs could decrease endotoxin-triggered vascular leakage.

### Example 22

Cofilin and its Ser-3-phos-phorylated levels in Ad.Lcn10-infected ECs and control cells were measured by Western-blotting. We observed that Ad.Lcn10-ECs displayed higher levels of Ssh1 than Ad.GFP-cells. The phosphorylated levels of Cofilin were significantly lower in Ad.Lcn10-cells than in Ad.GFP-cells, suggesting its dephosphorylation and activation by Ssh1. Given that Cofilin is well known to control actin filament reorganization by stimulating the severance and depolymerization of actin filaments, we next examined cytoskeletal structures (i.e., stress fibers and cortical actin networks), using Alexa-Fluor 594 phalloidin (F-actin probe) staining. Under basal conditions, GFP-ECs showed a cortical organization of cytoskeletal F-actin along the cell periphery. After LPS stimulation, peripherally located F-actin is substituted by centralized and irregular stress fibers running across the cytoplasm. However, overexpression of Lcn10 strongly reinforced peripheral actin bands, both in unstimulated and LPS-treated cells. Importantly, LPS-triggered formation of stress fibers was less pronounced in Len10-cells than in control GFP-cells. Given that reorganization of the actin cytoskeleton affects the stability of EC junctions and paracellular gap formation, we lastly evaluated the integrity of cell peripheral membrane by staining these ECs with antibody to ZO-1, a tight junction protein. The linear shape of cell-cell junctions was displayed in both GFP- and Lcn10-cells under basal conditions. However, LPS treatment greatly impaired the integrity of cell membranes, as evidenced by jagged and disconnected ZO-1 staining in GFP-cells, but not in Lcn10-cells. Together, these data indicate that forced elevation of Lcn10 in ECs activates the Ssh1-Cofilin pathway and reinforces the cortical actin network upon LPS insult, leading to reduced stress fiber formation and opening of cell junctions.

### Example 23

To test whether Lcn10-elicited action is dependent on Ssh1 signaling, sequence-specific siRNAs were first utilized to knockdown Ssh1 expression in Ad.Lcn10-infected ECs, followed by LPS challenge for EC permeability analysis. Western-blotting data revealed that elevation of Ssh1 in Ad.Lcn10-ECs was greatly reduced by siRNA-Ssh1 to a similar level as in GFP-cells. Consequently, Lcn10-induced decrease in the levels of phosphorylated Cofilin was greatly blocked by siRNA-Ssh1 transfection, as revealed by a similar degree of Cofilin phosphorylation between siRNA-Ssh1/Ad.Lcn10-ECs and control siRNA-Ssh1/Ad.GFP-cells.

### Example 24

Given that rLcn10 upregulates endothelial Ssh1 expression, we next tested whether rLcn10-elicited decrease of EC monolayer leakage is dependent on Ssh1. ECs were transfected with siRNA-Ssh1 (Si-Ssh1) or siRNA-control (Si-Con) for 48 h, followed by addition of rLcn10 (200 ng/ml) or BSA control. Then, EC leakage was measured at 2 h post-LPS challenge. We observed that rLcn10-induced reduction of EC permeability was significantly blocked by siRNA-Ssh1 transfection, as evidenced by similar degrees of TEER and FITC/EB dye flux between BSA- and rLcn10-treated groups. Consistently, rLcn10-induced inhibition of stress fiber formation was greatly suppressed by Ssh1 knockdown. These data indicate that Ssh1 plays an essential role in Lcn10-induced benefits in reducing vascular leakage during sepsis.

### Example 25

To test if exogenous rLcn10 protein can rescue such a leakage in Lcn10-KO cells, we added rLcn10 (200 ng/ml) or control BSA to cultured cardiac ECs that were isolated from WT and Lcn10-KO mice, followed by permeability analysis at 2 h post-LPS insult. Our results showed that the leakage of FITC-dextran and EB-dye in KO-ECs was greatly inhibited by addition of rLcn10, compared to BSA-cells. These data indicate that rLcn10 can be used as an effective agent to block vascular leak during sepsis. In conclusion, loss of Len10 exacerbated sepsis-induced systemic and local inflammation, aggravated multiple organ injury and reduced the survival rate of polymicrobial septic mice. One crucial factor seemed to be the impaired phagocytic function of Lcn10 KO macrophages. The present invention presents the first evidence on the correlation between Lcn10 and phagocytosis/autophagy pathway in macrophages.

### Example 26

Type 2 diabetes (T2D) was induced in wild-type (WT) and Lcn10 knockout (KO, C57BL6 background) mice by combination of high-fat diet (HFD, 60%) feeding and streptozotocin (STZ, 100 mg/kg body weight) injection. Flow cytometry was performed to characterize cardiac immune cell composition and phenotype. The expression levels of various macrophage marker genes were measured by qPCR. Bone marrow-derived macrophages (BMDMs) were isolated from WT and Lcn10-KO mice to test macrophage function under stress conditions.

When BMDMs were treated with palmitate, it was observed that Lcn10 gene expression was significantly downregulated. Similar result was noted in BMDMs treated with high glucose (25 mM), compared to low glucose (5 mM) group. Consistently, Lcn10 gene expression was 40% lower in cardiac macrophages from diabetic mice, comparing to those from mice fed with chow diet (CD). These results indicate that Lcn10 may play a role in regulating macrophage function. To test this hypothesis, we treated WT and Lcn10-KO BMDMs with palmitate and assessed phenotypic changes using qPCR. The results showed that Lcn10-KO BMDMs had significantly higher expression of pro-inflammatory marker genes (i.e. iNOS, IL-6 and CCL2) in response to palmitate treatment, compared to WT group. Importantly, the macrophages in the hearts of Lcn10-KO diabetic (KO-T2D) mice exhibited stronger pro-inflammatory phenotype, as evidenced by higher ratio of Ly6C+ population to CD206+ population, compared to WT-T2D mice. Consequently, this led to reduced cardiac contractile function (10% decrease in ejection fraction, n=6-8, p<0.05). Mechanistically, RNA sequencing analysis using WT and Lcn10-KO BMDMs suggested that loss of Lcn10 disrupted Nr4a1 signaling pathway, resulting in downregulation of Nr4a1-targeted genes (i.e. CX3CR1, GDF3, MID1). Accordingly, Lcn10-KO BMDMs failed to respond to Nr4a1 agonist, which showed strong anti-inflammatory effects in WT BMDMs.

### Example 27

Sepsis models were induced in mice by both intraperitoneal (IP) injection of endotoxin LPS (10 mg/kg body weight) and cecal ligation and puncture (CLP). Using RT-qPCR analysis, we observed that Lcn10 was significantly down-regulated in the hearts of both LPS- and CLP-treated mice, compared to their controls. Interestingly, further analysis of Lcn10 expression in different cell types isolated from LPS- and CLP-treated hearts showed that reduction of Lcn10 occurred only in cardiac endothelial cells (ECs) but not in cardiomyocytes or fibroblasts. These data suggest that Lcn10 may be involved in sepsis-induced cardiovascular leakage. Using a global Lcn10-knockout (KO) mouse model, we found that loss of Lcn10 greatly increased vascular permeability, which correlated with more severe cardiac depression and higher mortality following LPS challenge or CLP surgery, compared to LPS- or CLP-treated wild-type (WT) mice. By contrast, in vitro overexpression of Lcn10 in ECs provided greater resistance to LPS-caused monolayer leak, compared to control cells. A mechanistic analysis by RNA-sequencing and RT-qPCR revealed that both endogenous and exogenous elevation of Len10 in ECs could significantly upregulate slingshot homolog 1 (Ssh1) expression. Ssh1 is a phosphatase known to activate Cofilin, a key actin-binding protein that plays an essential role in controlling actin filament dynamics. Accordingly, phosphorylated Cofilin levels were significantly reduced and thereby, reorganized F-actin to cortical actin for stabilizing tight junction molecules in Lcn10-treated ECs, compared to control cells. Finally, knockdown of Ssh1 in ECs by siRNA greatly offsets Lcn10-induced reduction of monolayer leakage upon LPS insult.

### Example 28

Since Len10 is a secreted protein, a global Lcn10-KO mouse model was generated. After co-culturing BMDMs with GFP-labeled dead myocytes for 2h, a 25% reduction on efferocytosis by KO-BMDMs, compared to wild-type (WT) controls was observed. Using Lcn10-KOmCherry mouse model (cardiac-specific overexpression of mCherry in Lcn10-KO mice), it was detected that KO cardiac macrophages ingested fewer dead myocytes at 3 days post-I/R, compared to WT-mCherry controls. Accordingly, cardiac contractile function, measured by echocardiography at the same time, was significantly declined in KO-mice, together with increased accumulation of apoptotic cells and higher serum levels of troponin I, a marker of cardiac damage, compared with WTs (n=7-10, p<0.05). In addition, pre-treatment of KO-BMDMs with recombinant Lcn10 protein could rescue the impaired efferocytosis. Mechanistically, RNA-seq analysis showed that many downregulated genes (i.e., ItgaV, Itga6, Cx3cr1, and Msr) in KO-macrophages are associated with efferocytosis. Interestingly, it was identified that Lcn10 contains a potential phosphatidylserine (PS)-binding motif (RxKRK), located at a helix-turn-helix structure, which is the primary “find-me” signal for efferocytosis.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of reducing the risk of a sepsis-induced vascular leak, tissue edema or organ dysfunction in a subject comprising administering an effective amount of a composition selected from the group consisting of Lipocalin 10 (SEQ ID NO: 1), a truncated Lipocalin 10 (Lcn10) protein having the amino acid sequence SEQ ID NO: 2, Lcn10-expressing vectors for full length/truncated Lcn10, and combinations thereof to said subject.

2. The method of claim 1 wherein the method reduces the risk of a sepsis-induced vascular leak.

3. The method of claim 1 wherein the method reduces the risk of tissue edema.

4. The method of claim 1 wherein the method reduces the risk of organ dysfunction.

5. The method of claim 1 wherein the subject is administered Lipocalin 10 (SEQ ID NO:1).

6. The method of claim 1 wherein the subject is administered a truncated Lipocalin 10 protein having the amino acid sequence SEQ ID NO: 2.

7. The method of claim 1 wherein the subject is administered with Lipocalin 10 (SEQ ID NO: 1) or a truncated Lipocalin 10 (SEQ ID NO: 2) at a dosage of 50-200 ng/g body weight.

8. The method of claim 7 wherein the subject is administered with Lipocalin 10 (SEQ ID NO: 1) or a truncated Lipocalin 10 (SEQ ID NO: 2) via vein injection.

9. A method of reducing the risk of a heart attack-induced cardiac dysfunction, atherosclerosis, inflammatory bowel disease or diabetes-induced cardiomyopathy in a subject comprising administering an effective amount of a composition selected from the group consisting of Lipocalin 10 (SEQ ID NO: 1), a truncated Lipocalin 10 (Lcn10) protein having the amino acid sequence SEQ ID NO: 2, Lcn10-expressing vectors for full length/truncated Lcn10, and combinations thereof to said subject.

10. The method of claim 9 wherein the method reduces the risk of a heart attack-induced cardiac dysfunction.

11. The method of claim 9 wherein the method reduces the risk of atherosclerosis.

12. The method of claim 9 wherein the method reduces the risk of inflammatory bowel disease.

13. The method of claim 9 wherein the method reduces the risk of diabetes-induced cardiomyopathy.

14. The method of claim 9 wherein the subject is administered Lipocalin 10 (SEQ ID NO:1).

15. The method of claim 9 wherein the subject is administered a truncated Lipocalin 10 protein having the amino acid sequence SEQ ID NO: 2.

16. The method of claim 9 wherein the subject is administered with Lipocalin 10 (SEQ ID NO: 1) or a truncated Lipocalin 10 (SEQ ID NO: 2) at a dosage of 50-200 ng/g body weight.

17. The method of claim 16 wherein the subject is administered with Lipocalin 10 (SEQ ID NO: 1) or a truncated Lipocalin 10 (SEQ ID NO: 2) via vein injection.

18. A pharmaceutical composition comprising a truncated Lipocalin 10 protein having the amino acid sequence SEQ ID NO: 2.

19. The pharmaceutical composition of claim 18 wherein the composition is for use in a method of reducing the risk of a sepsis-induced vascular leak, tissue edema or organ dysfunction in a subject.

20. The pharmaceutical composition of claim 18 wherein the composition is for use in a method of reducing the risk of a heart attack-induced cardiac dysfunction, atherosclerosis, inflammatory bowel disease or diabetes-induced cardiomyopathy in a subject.


SEQUENCE LISTING (2 sequences)

SEQ ID NO: 1 - protein, 187 aa, Homo sapiens
MRQGLLVLALVLVLVLVLAAGSQVQEWYPRESHALNWNKFSGFWYILATATDAQGFLPARDKRKLGASVVKVNKVGQLRVLLAFRRGQGCGRAQPRHPGTSGHLWASLSVKGVKAFHVLSTDYSYGLVYLRLGRATQNYKNLLLFHRQNVSSFQSLKEFMDACDILGLSKAAVILPKDASRTHTILP

SEQ ID NO: 2 - protein, 121 aa, Homo sapiens
AGSQVQEWYPRESHALNWNKFSGFWYILATATDAQGFLPARDKRKLGASVVKVNKVGQLRVLLAFRRGQGCGRAQPRHPGTSGHLWASLSVKGVKAFHVLSTDYSYGLVYLRLGRATQNYK